(19) United States
(12) Patent Application Publication
(10) Pub. No.: US 2020/0015702 A1
(43) Pub. Date: Jan. 16, 2020

(54) SYSTEM AND METHOD FOR LANDMARKING A PATIENT

(71) Applicant: Children's Hospital Medical Center, Cincinnati, OH (US)

(72) Inventors: Charles Lucian DUMOULIN, Cincinnati, OH (US); Steven Gerard LEE, Mason, OH (US); Christoph Lewis GILLUM, Mason, OH (US)

(73) Assignees: Children's Hospital Medical Center, Cincinnati, OH (US); NeoView, Inc., Cincinnati, OH (US)

(21) Appl. No.: 16/506,334

(22) Filed: Jul. 9, 2019

Related U.S. Application Data
(60) Provisional application No. 62/697,420, filed on Jul. 13, 2018.

(51) Int. Cl. A61B 5/055; A61B 90/00

(52) U.S. Cl. CPC A61B 5/0555; A61B 2090/3762; A61B 90/37

(57) ABSTRACT

A system for landmarking a patient during a medical procedure, such as Magnetic Resonance (MR) Imaging, is disclosed. A video display monitor displays images from a video camera positioned in a location relative to an isocenter of the medical device, and a patient's position is adjusted to align a feature of interest on the patient with a reference marker displayed on the monitor. A landmark may be declared on the feature of interest such that the system can move the patient to place the landmarked feature of interest substantially at the isocenter of the medical device.

### CROSS REFERENCE TO RELATED APPLICATIONS

The current application claims priority from U.S. Provisional Application No. 62/697,420, filed Jul. 13, 2018, the entire enclosure of which is incorporated herein by reference.

### FIELD OF THE INVENTION

The present invention relates generally to landmarking of a patient undergoing a medical procedure, and more particularly, to a system for establishing patient landmarks in Magnetic Resonance (MR) and Computed Tomography (CT) systems without the need for a light source such as a laser, fiducial markers, or mechanical indicators.

### BACKGROUND OF THE INVENTION

The following background includes information that may be useful in understanding the present subject matter. It is not an admission that any of the information provided herein is prior art or relevant to the presently claimed subject matter, or that any publication specifically or implicitly referenced is prior art.

MR imaging of internal body tissues may be used for numerous medical procedures, including diagnosis and image guidance during surgery. In general terms, Magnetic Resonance Imaging (MRI) employs a relatively uniform, static magnetic field to polarize the spin magnetization in a patient's body. The spin magnetization that is most often used in MRI arises from the nuclei of hydrogen atoms within the body. Although the highest concentration of hydrogen atoms within the body is found in water molecules, other compounds found in the body (e.g. lipids, glucose, etc.) are present in sufficient concentration to provide a detectable MR spin magnetization.

The static magnetic field causes hydrogen nuclei spins to align and precess about the general direction of the magnetic field. Radio frequency (RF) magnetic field pulses are then superimposed on the static magnetic field to cause the aligned spins to alternate between a high-energy non-aligned state and the aligned state, thereby inducing an RF response signal, called the MR echo or MR response signal. The ratio of the number nuclear spins in the aligned verses non-aligned state is defined by the Boltzmann equation:

$$\frac{\text{Number of spins in the excited state}}{\text{Number of spins in the ground state}} = exp ( \frac{- ( {E}_{excited} - {E}_{ground} )}{kT} ) \qquad [ 1 ]$$

where the ratio of spins in the excited to ground state represents the spin polarization, Eexcited is the energy level of the excited state, E_{ground} is the energy of the ground state, T is temperature and k is the Boltzmann constant. This ratio defines the overall strength of the observable MR response signal.

It is known that different tissues in the subject produce different MR response signals, and this property can be used to create contrast in an MR image. An RF receiver detects the duration, strength, and source location of the MR response signals, and such data are then processed to generate tomographic or three-dimensional images.

MR imaging can also be used effectively during a medical procedure to assist in locating and guiding medical instruments. For example, a medical procedure can be performed on a patient using medical instruments while the patient is in an MRI scanner. The medical instruments may be for insertion into a patient or they may be used externally but still have a therapeutic or diagnostic effect. For instance, the medical instrument can be an ultrasonic device, which is disposed outside a patient's body and focuses ultrasonic energy to ablate or necrose tissue or other material on or within the patient's body. The MRI scanner preferably produces images at a high rate so that the location of the instrument (or the focus of its effects) relative to the patient may be monitored in real-time (or substantially in real-time). The MRI scanner can be used for both imaging the targeted body tissue and locating the instrument, such that the tissue image and the overlaid instrument image can help track an absolute location of the instrument as well as its location relative to the patient's body tissue.

Computed Tomography employs an X-ray source and X-ray detector that is rotated around the patient. The X-rays passing through the patient from a variety of directions are detected and measures of X-ray intensity are sent to a reconstruction algorithm which produces a cross-sectional image whose information content reflects the X-ray attenuation properties of tissue.

Precise positioning of the patient inside of an MR magnet or CT gantry is critical since each modality acquires images from the isocenter of the magnet or gantry. Traditionally, patient placement is accomplished with a landmarking laser cross-hair that is positioned a known distance from the imaging isocenter in the direction of the patient table motion. Before insertion into the imaging gantry the patient is moved under the landmarking laser until the laser cross-hair falls on, or near, the region of anatomy of interest. The operator then declares a landmark, and the imaging system brings the anatomy to the imaging gantry center.

The landmarking procedure described above requires the use of visible light. The operator needs to be able to see both the patient and the laser cross-hair. In some circumstances, for example when landmarking a sleeping baby or child, the requirement for room light can disturb the patient's sleep. Furthermore, the use of lasers for landmarking can potentially harm patients, especially if the laser cross-hair falls on the eyes of the patient. In view of the foregoing, it may be understood that the ability to establish landmarks on patients without the requirement for laser cross-hairs or room lights may be desirable and advantageous under some circumstances.

### SUMMARY

Embodiments of the present invention provide a landmarking system that may be useful in Magnetic Resonance Imaging (MRI), Computed Tomography (CT), gamma camera, Positron Emission Tomography (PET), and Radiation Therapy (RT) systems. In one embodiment, a video camera mounted in the ceiling is positioned such that a patient lying on the imaging or RT system table is within the camera's field of view. The video image acquired by the camera is sent to a display monitor, typically disposed near the patient and easily seen by the system operator. The operator can then move the patient towards the gantry and when the patient is appropriately positioned, as visualized by the operator on the display monitor, the operator depresses a landmark button thereby declaring a landmark. The location of the desired landmark can be depicted on the video display as a line or cross-hair superimposed upon the video image. Alternatively, a conventional laser cross-hair or other optical marker can be shined on the patient and visualized on the video monitor. Note that in this embodiment the laser need not be directly visible to the operator as long as it is detectable by the video camera. Once the landmark has been declared, the operator moves the patient to the isocenter of the imaging system using a second button, switch or lever. It should be noted that in the present invention the operator can be a natural person, or an artificial intelligence system configured to act like a natural person within the extent of patient landmarking process disclosed here. It should also be noted that in the present invention the video camera can be defined as any system that can capture a scene in one location and reproduce it at another location. Such devices include analog and digital electronic cameras, mirrors, periscopes, and the like.

In one particular exemplary embodiment, the imaging system is an MRI system used for diagnostic imaging of sleeping babies. In such a system the infrared sensitivity of the ceiling-mounted camera is used to visualize the patient in near or total darkness.

In another particular exemplary embodiment, the display monitor is situated in the operator control room and is not physically next to the patient. This arrangement allows the operator to landmark a patient without being in the same room as the diagnostic imaging or RT system.

The present invention will now be described in more detail with reference to exemplary embodiments thereof as shown in the accompanying drawings. While the present invention is described below with reference to exemplary embodiments, it should be understood that the present invention is not limited thereto. Those of ordinary skill in the art having access to the teachings herein will recognize additional implementations, modifications, and embodiments, as well as other fields of use, which are within the scope of the present invention as described herein, and with respect to which the present invention may be of significant utility.

### BRIEF DESCRIPTION OF THE DRAWINGS

In order to facilitate a fuller understanding of the present invention, reference is now made to the accompanying drawings, in which like elements are referenced with like numerals. These drawings should not be construed as limiting the present invention, but are intended to be exemplary only.

FIG. 1 shows an exemplary medical system in or for which the techniques for patient landmarking using a video camera in accordance with the present invention may be implemented.

FIG. 2a shows an exemplary display of the body of a patient prior to landmarking in accordance with an embodiment of the present invention.

FIG. 2b shows an exemplary display of the body of a patient after landmarking in accordance with an embodiment of the present invention.

FIG. 3 depicts an example computing environment in which embodiments of the invention can be implemented.

### DETAILED DESCRIPTION

Embodiments of the present invention provide apparatus(es) and/or method(s) to landmark a patient for diagnostic imaging and/or radiation therapy.

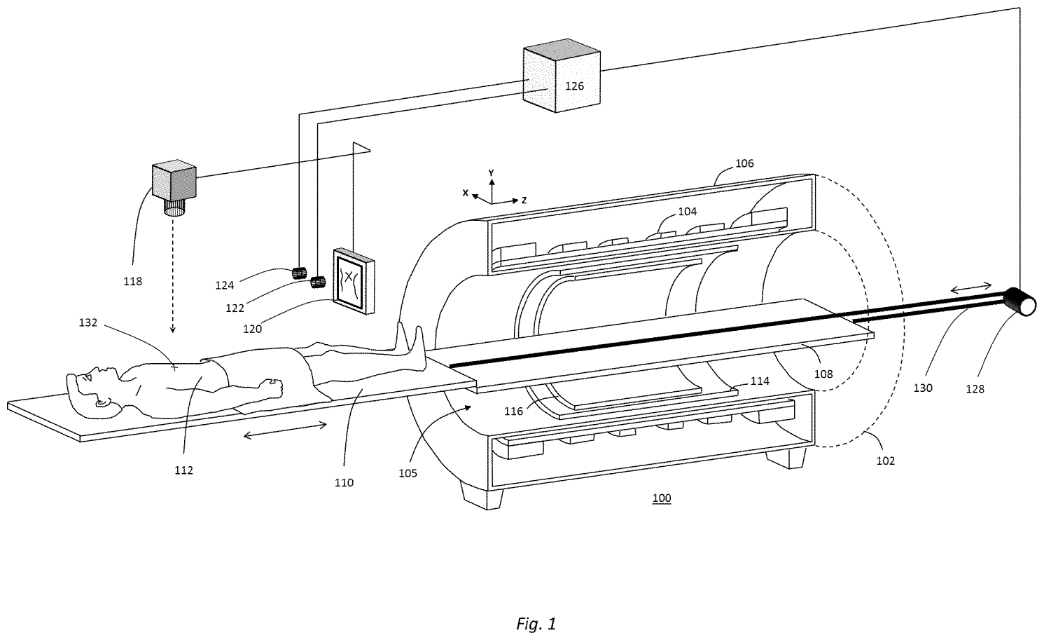

FIG. 1 shows an exemplary medical system 100 in or for which the techniques patient landmarking in accordance with the present invention may be implemented. While FIG. 1 will be discussed in terms of a Magnetic Resonance Imaging (MRI) system, it will be noted that embodiments are not limited to MRI systems. Other medical systems on which the presented invention may be utilized include Computed Tomography (CT), Gamma Camera, Positron Emission Tomography (PET), and Radiation Therapy (RT) systems. The illustrated MRI system 100 comprises an MRI scanner 102. Since the components and operation of the MRI scanner are well-known in the art, only some basic components helpful in the understanding of the system 100 and its operation will be described herein.

The MRI scanner 102 typically comprises a cylindrical superconducting magnet 104, which generates a static magnetic field within a bore 105 of the superconducting magnet 104. The superconducting magnet 104 generates a substantially homogeneous magnetic field within an imaging region inside the magnet bore 105. The superconducting magnet 104 may be enclosed in a magnet housing 106. A support table 108, upon which a patient table 110 lies, is disposed within the magnet bore 105. Patient table 110 is configured to slide into and out of MRI scanner 102. A patient 112 is positioned on top of patient table 110 in a supine, prone, or other orientation. The intent of the present invention is to identify a desired region of interest within patient 112 and position it within the imaging region of the MRI scanner 102 which is typically the center of the superconducting magnet 104.

A set of cylindrical magnetic field gradient coils 114 may also be provided within the magnet bore 105. The gradient coils 114 also surround the patient 112. The gradient coils 114 can generate magnetic field gradients of predetermined magnitudes, at predetermined times, and in three mutually orthogonal directions within the magnet bore 105. With the field gradients, different spatial locations can be associated with different precession frequencies, thereby giving an MR image its spatial resolution. An RF transmitter coil 116 surrounds the imaging region. The RF transmitter coil 116 emits RF energy in the form of a rotating magnetic field into the imaging region.

The RF transmitter coil 116 can also receive MR response signals emitted from the region of interest. The MR response signals are amplified, conditioned and digitized into raw data using an image processing system, as is known by those of ordinary skill in the art. The image processing system further processes the raw data using known computational methods, including fast Fourier transform (FFT), into an array of image data. The image data may then be displayed on a monitor, such as a computer CRT, LCD display or other suitable display.

To make MR images of the anatomy of interest in patient 112 it is desired to put the anatomy of interest in the center of superconducting magnet 104, gradient coils 114, and RF transmitter coil 116. In the present invention this is accomplished using a video camera 118 that can capture a video image of patient 112 outside of MRI scanner 102. Video camera 118 can be mounted in the ceiling above patient 112, on MRI scanner 102, or placed in any known location near patient 112 or the medical device on which it is used. The video image captured by video camera 118 is displayed on one or more display monitors 120. Display monitors 120 can be disposed near the opening of magnet bore 105 as shown in FIG. 1, in a same or different room as the medical device, or in any convenient location including the system control room. In embodiments, the control room can be remotely located. The system operator moves patient 112 towards magnet bore 105 until the video image of the desired patient anatomy is appropriately positioned. The desired patient anatomy can be identified by a superficial feature 132 which can be a naturally occurring anatomic feature, a marking made by the operator or other medical staff, or an object of interest such as an item of clothing or MR imaging coil. It should be noted that display monitor 120 can provide a black and white, color, or holographic image to the operator.

In the embodiment illustrated in FIG. 1, movement of patient 112 is accomplished by activating a motor 128 which pulls on a belt 130 which is attached to patient table 110. Pulling belt 130 causes patient table 110 to roll onto support table 108. Motor 128 is controlled by a set of system control electronics 126 which is also attached to a table movement actuator 124 and a landmark declaration button 122.

Note that a variety of implementations of table movement actuator 124 are possible including buttons, switches and levers. Likewise, a variety of table movement mechanisms including electromagnetic drives, screw drives, chains, and the like fall within the spirit of the invention. Likewise, the declaration of a landmark need not be made with a button, but could be performed via an audible tone or physical gesture.

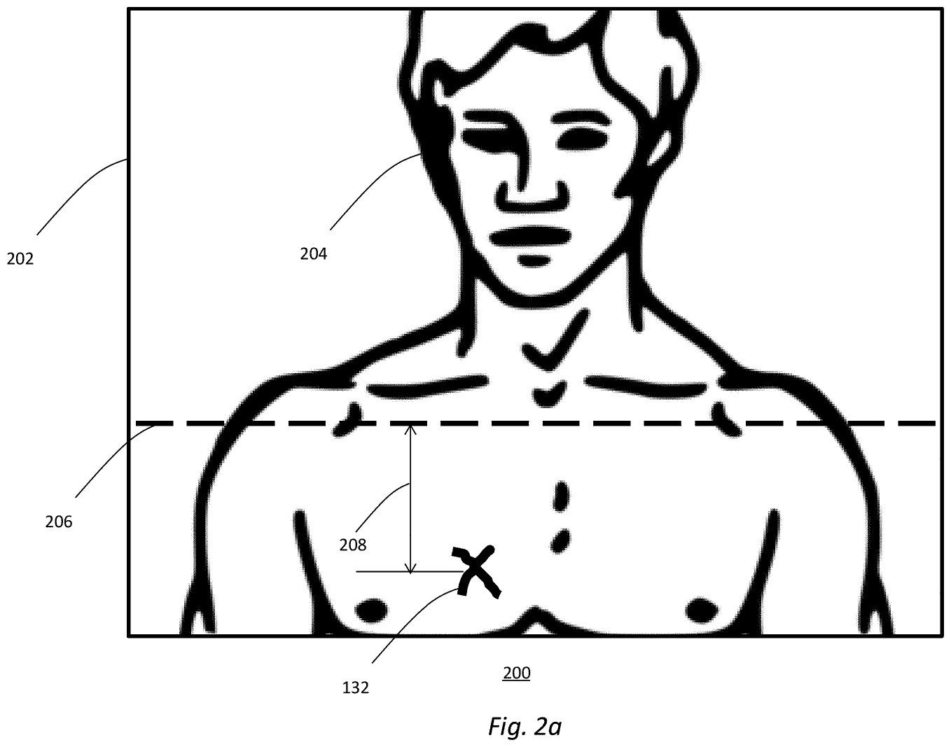

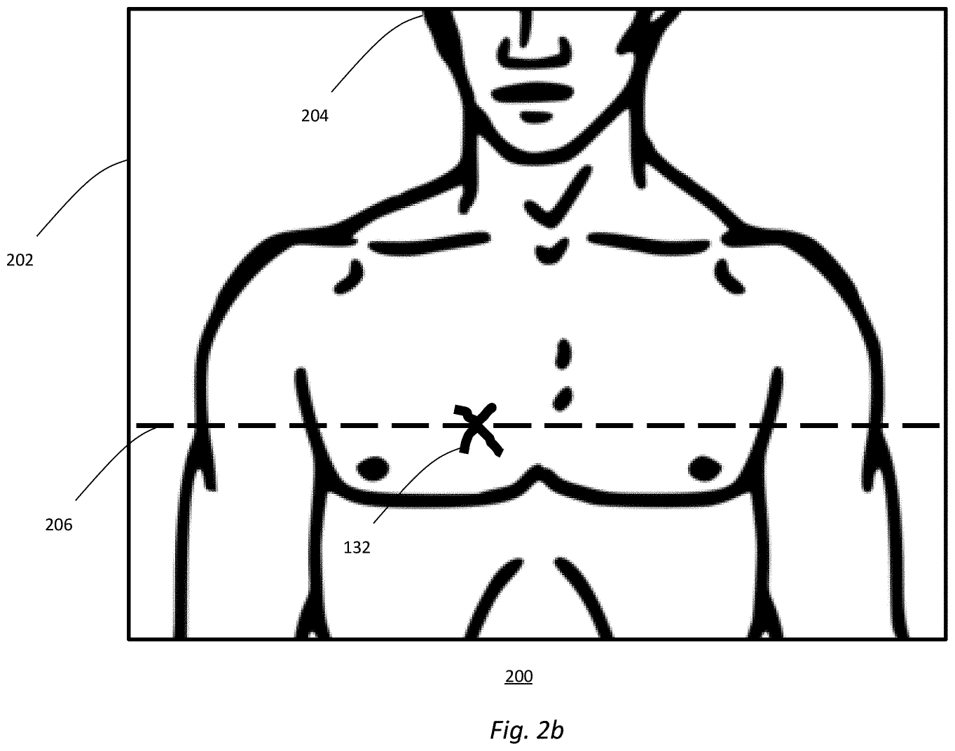

Superficial feature 132 is visualized by the operator on display monitor 120, and the operator moves patient 112 until superficial feature 132 is aligned with a reference mark 206 provided on display monitor 120, shown in greater detail in FIGS. 2a and 2b. The location of reference mark 206 on display monitor 120 is a known distance to the magnet isocenter. Once the operator is satisfied that superficial feature 132 is properly aligned with reference mark 206, on display monitor 120 the operator presses landmark declaration button 122. Once the landmark has been declared, system control electronics 126 are invoked to cause motor 128 to pull patient table 110 until superficial feature 132 is located at the center of magnet 104. The operator can then proceed to scan the patient.

FIG. 2 illustrates in greater detail exemplary contents 200 of display monitor 120 during the landmarking process. FIG. 2a shows a video presentation 202 in which superficial feature 132 is not in alignment with reference mark 206. As the operator moves patient 112, video presentation 202 shows a patient image 204 moving within the field of view of video camera 118. The operator then moves patient 112 a selected distance 208 to bring the video representation of superficial feature 132 into alignment with reference mark 206 as shown in FIG. 2b.

Note that reference mark 206 can be a physical mark on the screen of display monitor 120, a video overlay placed on top of patient image 204. For example, the reference mark may comprise a video signal mixed with a video of the patient, to provide a composite image on the video display monitor. Alternatively, reference mark 206 could be a laser line or cross-hair or other optical marker placed on patient 112 which is captured by video camera 118 and presented on the at least one video display monitor. In other embodiments, the reference mark can be part of the at least one video display monitor. It is noteworthy that in this particular embodiment the laser need not be visible to the human eye, and that in other embodiments, the reference mark may be a visible mark. The video camera can also be sensitive to wavelengths of light that are beyond human perception. As long as the laser has a wavelength within the spectral sensitivity band of video camera 118, it will appear as a visible marker on display monitor 120 and can be used for landmarking purposes. For example, in one particular exemplary embodiment, the imaging system is an MRI system used for diagnostic imaging of sleeping babies. In such a system, the laser is an infrared laser, and infrared sensitivity of the ceiling-mounted camera 118 is used to visualize the laser marking and/or the patient in near or total darkness. In another embodiment, the ceiling-mounted camera is a night-vision camera as commercially available to those having ordinary skill, where the night-vision camera is capable of displaying patient features 132 on the video screen 120 for landmarking, while the patient is lying is substantial darkness.

A salient feature of the present invention is that it permits landmarking of patients without requiring the operator to see the patient directly. That is, one or more aspects of the present invention, including aligning the superficial feature of interest with the reference marker, declaring a landmark, and moving the patient to the isocenter of the medical device, can be performed without using visible light to illuminate the patient. Consequently, the landmarking process can be safely accomplished in reduced light settings and in total darkness. The present invention also permits the operator to establish a landmark without being in the room with the patient. This may be useful in situations in which the diagnostic imaging or radiation therapy equipment is operated remotely. It also anticipates the use of artificially intelligent software systems that are capable of acting like an operator in detecting superficial features of interest for landmarking. In embodiments, the artificial intelligent software systems can operate on one or more computing systems and networks, and utilize neural networks and training algorithms to at least automate one or more aspects of the landmarking system and methods described herein and/or optimize placement of the landmark on the patient.

Another salient feature of the present invention is that it does not require the use of any fiducial markers to identify the landmark location. Identification of the landmark is under the operator's control and judgement.

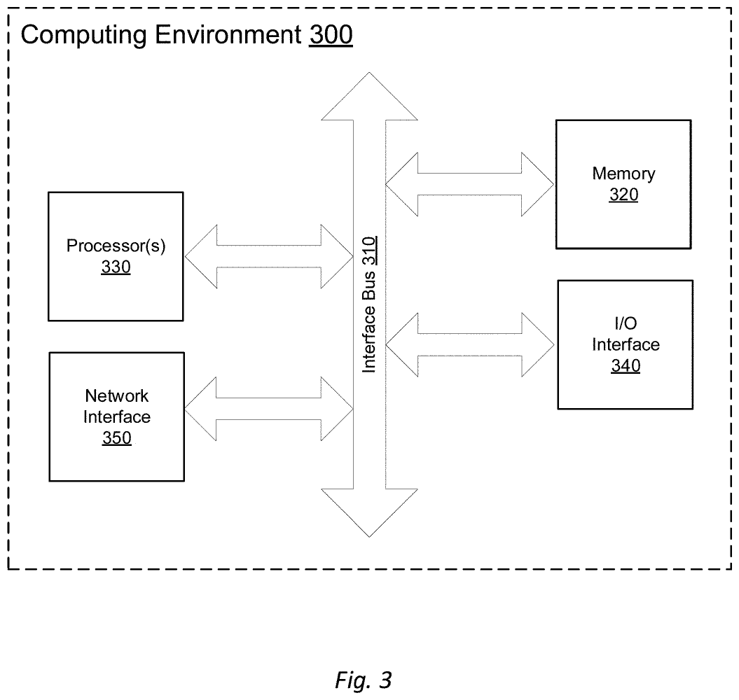

Having briefly described an overview of embodiments of the invention, an example of a computing environment suitable for implementing aspects of the embodiments is described below. Referring to the figures generally and initially to FIG. 3 in particular, an exemplary computing environment in which embodiments and aspects of the present invention is depicted and generally referenced as computing environment 300. As utilized herein, the phrase “computing system” generally refers to a dedicated computing device with processing power and storage memory, which supports operating software that underlies the execution of software, applications, and computer programs thereon. As shown by FIG. 3, computing environment 300 includes bus 310 that directly or indirectly couples the following components: memory 320, one or more processors 330, I/O interface 340, and network interface 350. Bus 310 is configured to communicate, transmit, and transfer data, controls, and commands between the various components of computing environment 300.

Computing environment 300 typically includes a variety of computer-readable media. Computer-readable media can be any available media that is accessible by computing environment 300 and includes both volatile and nonvolatile media, removable and non-removable media. Computer-readable media may comprise both computer storage media and communication media. Computer storage media does not comprise, and in fact explicitly excludes, signals per se.

Computer storage media includes volatile and nonvolatile, removable and non-removable, tangible and non-transient media, implemented in any method or technology for storage of information such as computer-readable instructions, data structures, program modules or other data. Computer storage media includes RAM; ROM; EE-PROM; flash memory or other memory technology; CD-ROMs; DVDs or other optical disk storage; magnetic cassettes, magnetic tape, magnetic disk storage or other magnetic storage devices; or other mediums or computer storage devices which can be used to store the desired information and which can be accessed by computing environment 300.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, communication media includes wired media, such as a wired network or direct-wired connection, and wireless media, such as acoustic, RF, infrared and other wireless media. Combinations of any of the above should also be included within the scope of computer-readable media.

Memory 320 includes computer-storage media in the form of volatile and/or nonvolatile memory. The memory may be removable, non-removable, or a combination thereof. Memory 320 may be implemented using hardware devices such as solid-state memory, hard drives, optical-disc drives, and the like. Computing environment 300 also includes one or more processors 330 that read data from various entities such as memory 320, I/O interface 340, and network interface 350.

I/O interface 340 enables computing environment 300 to communicate with different input devices and output devices. Examples of input devices include a camera, a keyboard, a pointing device, a touchpad, a touchscreen, a scanner, a microphone, a joystick, and the like. Examples of output devices include a display device, an audio device (e.g. speakers), a printer, and the like. These and other I/O devices are often connected to processor 310 through a serial port interface that is coupled to the system bus, but may be connected by other interfaces, such as a parallel port, game port, or universal serial bus (USB). A display device can also be connected to the system bus via an interface, such as a video adapter which can be part of, or connected to, a graphics processor unit. I/O interface 340 is configured to coordinate I/O traffic between memory 320, the one or more processors 330, network interface 350, and any combination of input devices and/or output devices.

Network interface 350 enables computing environment 300 to exchange data with other computing devices via any suitable network. In a networked environment, program modules depicted relative to computing environment 300, or portions thereof, may be stored in a remote memory storage device accessible via network interface 350. It will be appreciated that the network connections shown are exemplary and other means of establishing a communications link between the computers may be used.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

It is understood that the term circuitry used through the disclosure can include specialized hardware components. In the same or other embodiments circuitry can include microprocessors configured to perform function(s) by firmware or switches. In the same or other example embodiments circuitry can include one or more general purpose processing units and/or multi-core processing units, etc., that can be configured when software instructions that embody logic operable to perform function(s) are loaded into memory, e.g., RAM and/or virtual memory. In example embodiments where circuitry includes a combination of hardware and software, an implementer may write source code embodying logic and the source code can be compiled into machine readable code that can be processed by the general purpose processing unit(s). Additionally, computer executable instructions embodying aspects of the invention may be stored in ROM EEPROM, hard disk (not shown), RAM, removable magnetic disk, optical disk, and/or a cache of processing unit. A number of program modules may be stored on the hard disk, magnetic disk, optical disk, ROM, EEPROM or RAM, including an operating system, one or more application programs, other program modules and program data.

While the foregoing description includes many details and specificities, it is to be understood that these have been included for purposes of explanation only, and are not to be interpreted as limitations of the present invention. It will be apparent to those skilled in the art that other modifications to the embodiments described above can be made without departing from the spirit and scope of the invention. Accordingly, such modifications are considered within the scope of the invention as intended to be encompassed by the following claims and their legal equivalents.

What is claimed is:

1. A system for landmarking a patient in a medical device comprising:
a video camera positioned in a known location relative to an isocenter of said medical device;
at least one video display monitor suitable for displaying images acquired by the video camera;
a reference marker placed on the images acquired by the video camera and displayed on the video display monitor;
a means for moving the patient to align a superficial feature of interest with the reference marker as visualized on the at least one video display monitor;
a means for declaring a landmark; and
a means for moving the patient such that the superficial feature of interest is placed substantially at the isocenter of the medical device.

2. The system of claim 1, wherein the medical device is a Magnetic Resonance Imaging system.

3. The system of claim 1, wherein the medical device is a Computed Tomography system.

4. The system of claim 1, wherein the medical device is a Positron Emission Tomography system.

5. The system of claim 1, wherein the medical device is a Gamma Camera system.

6. The system of claim 1, wherein the medical device is a Radiation Therapy system.

7. The system of claim 1, wherein the video camera is mounted in a ceiling above the patient.

8. The system of claim 1, wherein the video camera is mounted directly on the medical device.

9. The system of claim 1, wherein the video camera is sensitive to wavelengths of light that are beyond human perception.

10. The system of claim 1, wherein the at least one video display monitor is placed in a same room as the medical device.

11. The system of claim 1, wherein the at least one video display monitor is placed in a different room as the medical device.

12. The system of claim 1, wherein the at least one video display monitor provides a holographic image.

13. The system of claim 1, wherein the reference marker is part of the at least one video display monitor.

14. The system of claim 1, wherein the reference marker is a video signal that is mixed with a video of the patient to provide a composite image on the at least one video display monitor.

15. The system of claim 1, wherein the reference marker is a laser or optical marker applied to the patient and captured by the video camera and presented on the at least one video display monitor.

16. The system of claim 15, wherein the laser or optical marker is not visible to a human eye, but is within a spectral sensitivity of the video camera and is presented on the at least one video display monitor as a visible marker.

17. The system of claim 1, wherein the means for moving the patient is comprised of a table that moves from a location outside of the medical device to the isocenter of the medical device.

18. A method for landmarking a patient in a medical device comprising:
placement of a video camera in a known location relative to an isocenter of said medical device;
placement of at least one video display monitor visible to an operator wherein said video display monitor is suitable for displaying images acquired by the video camera;
placement of a reference marker on the images acquired by the video camera and displayed on the video display monitor;
moving the patient to align a superficial feature of interest with the reference marker as visualized on the video display monitor;
declaring a landmark; and
moving the patient such that the superficial feature of interest is placed substantially at the isocenter of the medical device.

19. The method of claim 18 wherein the operator is an artificial intelligence system.

20. The method of claim 18, wherein the steps of moving the patient to align the superficial feature of interest with the reference marker, the declaration of a landmark, and the moving of the patient to the isocenter of the medical device, are performed substantially in darkness.

21. The method of claim 18, wherein the steps of moving the patient to align the superficial feature of interest with the reference marker, the declaration of a landmark, and the moving of the patient to the isocenter of the medical device, are performed without using visible light to illuminate the patient.

22. A system for landmarking a patient in a medical device comprising:
a video camera positioned in a known location relative to an isocenter of a medical device;
at least one video display monitor configured to display images acquired by the video camera;
a user interface associated with the video display monitor providing the ability for a user to landmark a feature of interest on the patient displayed by the video display monitor; and
a computer controlled mobile patient table configured to move the patient such that the landmarked feature of interest is placed substantially at the isocenter of the medical device.

23. The system of claim 22, wherein the video camera is one or more of an infrared camera or a night-vision camera.

24. The system of claim 23, wherein the feature of interest is an anatomical feature of the patient.

25. The system of claim 23, wherein the feature of interest is an infrared laser mark projected on the patient.
